(19) United States
(12) Patent Application Publication
(10) Pub. No.: US 2007/0099192 A1
(43) Pub. Date: May 3, 2007

(54) Denaturing size-fractionation in analysis of small RNA

(76) Inventor: Hui Wang, Palo Alto, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/264,783

(22) Filed: Oct. 31, 2005

(51) Int. Cl. C12Q 1/68

(52) U.S. Cl. 435/6

(57) ABSTRACT

The invention relates to analyzing a sample containing polynucleotides having small RNA binding sites. In exemplary embodiments, the sample is contacted with a size-fractionation medium under denaturing conditions to provide a fraction containing small RNA. The fraction containing small RNA is then contacted with an array under conditions sufficient to provide for specific binding to the array. The array is then interrogated to obtain information about small RNA in the sample. Kits in accordance with the invention are also described.

### RELATED APPLICATIONS

Related subject matter is disclosed in a U.S. patent application by Wang entitled “Microarray Analysis of RNA” under attorney docket number 10051298-1.

### DESCRIPTION

1. Field of the Invention

The invention relates generally to methods of biochemical analysis. More specifically, the invention relates to a method of analyzing a sample containing polynucleotides.

2. Background of the Invention

Since the discovery of the biological activity of short interfering RNAs (siRNAs) over a decade ago, so called “small RNAs” (i.e., short non-coding regulatory RNAs that have a defined sequence) have become a subject of intense interest in the research community. See Novina et al., Nature 430: 161-164 (2004). Exemplary small RNAs include siRNAs, microRNAs (miRNAs), tiny non-coding RNAs (tncRNAs) and small modulatory RNAs (smRNAs), as well as many others.

Although the exact biological functions of most small RNAs remain a mystery, it is clear that they are abundant in plants and animals, with up to tens of thousands of copies per cell. For example, to date, over 78 Drosophila microRNA species and 300 human microRNA species have been identified. The levels of the individual species of small RNA, in particular microRNA species, appear to vary according to the developmental stage and type of tissue being examined. It is thought that the levels of particular small RNAs may be correlated with particular phenotypes, as well as with the levels of particular messenger RNAs and proteins. Further, viral microRNAs have been identified, and their presence has been linked to viral latency (see Pfeffer et al., Science, 304: 734-736 (2004)).

The sequences of several hundred miRNAs from a variety of different species, including humans, may be found at the microRNA registry (Griffiths-Jones, Nucl. Acids Res. 2004 32:D109-D111), as found at the world-wide website of the Sanger Institute (Cambridge, UK) (which may be accessed by typing “www” followed by “.sanger.ac.uk/cgi-bin/Rfam/mirna/browse.pl” into the address bar of a typical internet browser). The sequences of all of the microRNAs deposited at the microRNA registry, including more than 300 microRNA sequences from humans (see Lagos-Quintana et al, Science 294:853-858(2001); Grad et al, Mol Cell 11:1253-1263(2003); Mourelatos et al, Genes Dev 16:720-728(2002); Lagos-Quintana et al, Curr Biol 12:735-739(2002); Lagos-Quintana et al, RNA 9:175-179(2003); Dostie et al, RNA 9:180-186(2003); Lim et al, Science 299:1540(2003); Houbaviy et al, Dev Cell 5:351-358(2003); Michael et al, Mol Cancer Res 1:882-891(2003); Kim et al, Proc Natl Acad Sci USA 101:360-365(2004); Suh et al, Dev Biol 270:488-498(2004); Kasashima et al, Biochem Biophys Res Commun 322:403-410(2004); and Xie et al, Nature 434:338-345(2005)), are incorporated herein by reference. MicroRNAs (miRNAs) are a class of single stranded RNAs of approximately 19-25 nt (nucleotides) in length.

Thus, analysis of miRNA may be of great importance, for example as a research or diagnostic tool. Analytic methods employing polynucleotide arrays have been used for investigating small RNAs, e.g. miRNAs have become a subject of investigation with microarray analysis. See, e.g., Liu et al., Proc. Nat'l Acad. Sci. USA, 101: 9740-9744 (2004); Thomson et al., Nature Methods, 1:1-7 (2004); and Babak et al., RNA, 10: 1813-1819 (2004). A considerable amount of effort is currently being put into developing array platforms to facilitate the analysis of small RNAs, particularly microRNAs. Polynucleotide arrays (such as DNA or RNA arrays) typically include regions of usually different sequence polynucleotides (“capture agents”) arranged in a predetermined configuration on a support. The arrays are “addressable” in that these regions (sometimes referenced as “array features”) have different predetermined locations (“addresses”) on the support of the array. The polynucleotide arrays typically are fabricated on planar supports either by depositing previously obtained polynucleotides onto the support in a site specific fashion or by site specific in situ synthesis of the polynucleotides upon the support. After depositing the polynucleotide capture agents onto the support, the support is typically processed (e.g., washed and blocked for example) and stored prior to use.

In use, an array is contacted with a sample (e.g. a labeled sample) containing analytes (typically, but not necessarily, other polynucleotides) under conditions that promote specific binding of the analytes in the sample to one or more of the capture agents present on the array. Thus, the arrays, when exposed to a sample, will undergo a binding reaction with the sample and exhibit an observed binding pattern. This binding pattern can be detected upon interrogating the array. For example all target polynucleotides (for example, DNA) in the sample can be labeled with a suitable label (such as a fluorescent compound), and the label then can be accurately observed (such as by observing the fluorescence pattern) on the array after exposure of the array to the sample. Assuming that the different sequence polynucleotides were correctly deposited in accordance with the predetermined configuration, then the observed binding pattern will be indicative of the presence and/or concentration of one or more components of the sample. Techniques for scanning arrays are described, for example, in U.S. Pat. No. 5,763,870 and U.S. Pat. No. 5,945,679. Still other techniques useful for observing an array are described in U.S. Pat. No. 5,721,435.

Straightforward and reliable methods for simultaneously analyzing several constituents of a complex RNA sample are extremely desirable. While current methods of preparing and analyzing RNA samples are quite useful, there is a continuing need for development of such methods.

### SUMMARY OF THE INVENTION

The invention thus relates to novel methods of performing an array analysis of an RNA sample. In certain embodiments, the invention provides a method of analyzing a sample containing polynucleotides having small RNA binding sites. The sample is contacted with a size-fractionation medium under denaturing conditions to provide a fraction containing small RNA. The fraction containing small RNA is then contacted with an array under conditions sufficient to provide for specific binding to the array. The array is then interrogated to obtain information about small RNA in the sample.

Additional objects, advantages, and novel features of this invention are set forth in part in the description follows and in part will become apparent to those skilled in the art upon examination of the following specifications or may be learned by the practice of the invention. The objects and advantages of the invention may be realized and attained by means of the instruments, combinations, compositions and methods particularly pointed out herein and in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1 schematically illustrates fractionation of a sample and also schematically illustrates hybridization of a sample to an array; and

FIG. 2 schematically illustrates an exemplary embodiment of the invention.

To facilitate understanding, identical reference numerals have been used, where practical, to designate corresponding elements that are common to the Figures. The Figure components are broadly illustrative and are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible in the present invention that steps may be executed in different sequence where this is logically possible. However, the sequence described below is preferred.

It must be noted that, as used in the specification and the appended claims, the singular forms “a,” “an” and “the” include plural referents unless the context clearly dictates otherwise. Thus, for example, reference to “an oligodeoxynucleotide” includes a plurality of oligodeoxynucleotides. Similarly, reference to “an RNA” includes a plurality of different identity (sequence) RNA species.

Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only,” and the like in connection with the recitation of claim elements, or use of a “negative” limitation. In this specification and in the claims that follow, reference will be made to a number of terms that shall be defined to have the following meanings unless a contrary intention is apparent.

“Optional” or “optionally” means that the subsequently described circumstance may or may not occur, so that the description includes instances where the circumstance occurs and instances where it does not. For example, if a step of a process is optional, it means that the step may or may not be performed, and, thus, the description includes embodiments wherein the step is performed and embodiments wherein the step is not performed (i.e. it is omitted).

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner similar to that of two naturally occurring nucleic acids, e.g., can participate in Watson-Crick base pairing interactions. An “oligonucleotide” is a molecule containing from 2 to about 100 nucleotides. The terms “nucleoside”, “nucleotide”, “oligonucleotide”, and “polynucleotide” are intended to include those moieties that contain the natural nucleotides (A, T, G, C, U), as well as those moieties that contain modified nucleotides, such as those in which the purine and pyrimidine bases have been modified or replaced with other heterocyclic bases. Such modifications include methylated purines or pyrimidines, acylated purines or pyrimidines, alkylated riboses or other heterocycles. In addition, the terms “nucleoside”, “nucleotide”, “oligonucleotide”, and “polynucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. Modified nucleosides or nucleotides also include molecules having structural features that are recognized in the literature as being mimetics, derivatives, having similar properties, or other like terms, and include, for example, polynucleotides incorporating non-natural (not usually occurring in nature) nucleotides, unnatural nucleotide mimetics such as 2′-modified nucleosides, peptide nucleic acids, oligomeric nucleoside phosphonates, and any polynucleotide that has added substituent groups, such as protecting groups or linking moieties.

“Sequence” may refer to a particular sequence of bases and/or may also refer to a polynucleotide having the particular sequence of bases. Thus a sequence may be information or may refer to a molecular entity, as indicated by the context of the usage. A duplex is a double stranded structure typically formed between complementary nucleic acid sequences. An intermolecular duplex is a double stranded structure typically formed between two different polynucleotide molecules that have complementary nucleic acid sequences, wherein the complementary nucleic acid sequences are hybridized to each other.

“Moiety” and “group” are used to refer to a portion of a molecule, typically having a particular functional or structural feature, e.g. a linking group (a portion of a molecule connecting two other portions of the molecule), or an ethyl moiety (a portion of a molecule with a structure closely related to ethane). A moiety is generally bound to one or more other moieties to provide a molecular entity. As a simple example, a hydroxyl moiety bound to an ethyl moiety provides an ethanol molecule. At various points herein, the text may refer to a moiety by the name of the most closely related structure (e.g. an oligonucleotide moiety may be referenced as an oligonucleotide, a mononucleotide moiety may be referenced as a mononucleotide). However, despite this seeming informality of terminology, the appropriate meaning will be clear to those of ordinary skill in the art given the context, e.g. if the referenced term has a portion of its structure replaced with another group, then the referenced term is usually understood to be the moiety. For example, a mononucleotide moiety is a single nucleotide which has a portion of its structure (e.g. a hydrogen atom, hydroxyl group, or other group) replaced by a different moiety (e.g. a linking group, an observable label moiety, or other group). Similarly, an oligonucleotide moiety is an oligonucleotide which has a portion of its structure (e.g. a hydrogen atom, hydroxyl group, or other group) replaced by a different moiety (e.g. a linking group, an observable label moiety, or other group). “Nucleotide moiety” is generic to both mononucleotide moiety and oligonucleotide moiety.

“Bound” may be used herein to indicate direct or indirect attachment. In the context of chemical structures, “bound” (or “bonded”) may refer to the existence of a chemical bond directly joining two moieties or indirectly joining two moieties (e.g. via a linking group or any other intervening portion of the molecule). The chemical bond may be a covalent bond, an ionic bond, a coordination complex, hydrogen bonding, van der Waals interactions, or hydrophobic stacking, or may exhibit characteristics of multiple types of chemical bonds. In certain instances, “bound” includes embodiments where the attachment is direct and also embodiments where the attachment is indirect. “Free,” as used in the context of a moiety that is free, indicates that the moiety is available to react with or be contacted by other components of the solution in which the moiety is a part.

“Isolated” or “purified” generally refers to isolation of a substance (compound, polynucleotide, protein, polypeptide, polypeptide, chromosome, etc.) such that the substance comprises a substantial portion of the sample in which it resides (excluding solvents), i.e. greater than the substance is typically found in its natural or un-isolated state. Typically, a substantial portion of the sample comprises at least about 2%, at least about 5%, at least about 10%, at least about 20%, at least about 30%, at least about 50%, at least about 80%, or at least about 90% of the sample (excluding solvents). For example, a sample of isolated RNA will typically comprise at least about 2% total RNA, or at least about 5% total RNA, where percent is calculated in this context as mass (e.g. in micrograms) of total RNA in the sample divided by mass (e.g. in micrograms) of the sum of (total RNA+other constituents in the sample (excluding solvent)). Techniques for purifying polynucleotides and polypeptides of interest are well known in the art and include, for example, gel electrophoresis, ion-exchange chromatography, affinity chromatography, and sedimentation according to density.

The term “sample” as used herein relates to a material or mixture of materials, typically, although not necessarily, in fluid form, containing one or more components of interest.

The term “analyte” is used herein to refer to a known or unknown component of a sample. In certain embodiments of the invention, an analyte may specifically bind to a capture agent on a support surface if the analyte and the capture agent are members of a specific binding pair. In general, analytes are typically RNA or other polynucleotides. Typically, an “analyte” is referenced as a species in a mobile phase (e.g., fluid), to be detected by a “capture agent” which, in some embodiments, is bound to a support, or in other embodiments, is in solution. However, either of the “analyte” or “capture agent” may be the one which is to be evaluated by the other (thus, either one could be an unknown mixture of components of a sample, e.g., polynucleotides, to be evaluated by binding with the other). A “target” references an analyte.

The term “capture agent” refers to an agent that binds an analyte through an interaction that is sufficient to permit the agent to bind and concentrate the analyte from a homogeneous mixture of different analytes. The binding interaction may be mediated by an affinity region of the capture agent. Representative capture agents include polypeptides and polynucleotides, for example antibodies, peptides, or fragments of double stranded or single-stranded DNA or RNA may employed. Capture agents usually “specifically bind” one or more analytes.

The terms “specific binding”, “specifically bind”, or other like terms, refers to the ability of a capture agent to preferentially bind to a particular analyte that is present in a homogeneous mixture of different analytes. In certain embodiments, a specific binding interaction will discriminate between desirable and undesirable analytes in a sample, in some embodiments more than about 10 to 100-fold or more (e.g., more than about 1000- or 10,000-fold). In certain embodiments, the binding constant of a capture agent and analyte is greater than 10⁶ M⁻¹, greater than 10⁷ M⁻¹, greater than 10⁸ M⁻¹, greater than 10⁹ M⁻¹, greater than 10¹⁰ M⁻¹, usually up to about 10¹² M⁻¹, or even up to about 10¹⁵ M⁻¹. “Specific binding conditions” are conditions sufficient to provide for a capture agent to preferentially bind to a particular analyte, e.g. stringent assay conditions.

The term “stringent assay conditions” as used herein refers to conditions that are compatible to produce binding pairs of nucleic acids, e.g., capture agents and analytes, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in Southern or Northern hybridizations, or hybridization of molecules in solution, or in array assays) are sequence dependent, and are different under different experimental conditions. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.1×SSC and 0.1% SDS at 37° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions may affect the degree to which nucleic acids are specifically hybridized to complementary capture agents. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 1 to about 20 minutes; or, multiple washes with a solution with a salt concentration of about 0.1×SSC containing 0.1% SDS at 20 to 50° C. for 1 to 15 minutes; or, equivalent conditions. Stringent conditions for washing can also be, e.g., 0.2×SSC/0.1% SDS at 42° C. In instances wherein the nucleic acid molecules are oligodeoxynucleotides (e.g. oligonucleotides made up of deoxyribonucleotide subunits), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “array” and the equivalent term “microarray” each reference an ordered array of capture agents for binding to aqueous analytes and the like. An “array” includes any two-dimensional or substantially two-dimensional (as well as a three-dimensional) arrangement of spatially addressable regions (i.e., “features”) containing capture agents, particularly polynucleotides, and the like. Any given support may carry one, two, four or more arrays disposed on a surface of a support. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 100 cm², 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of the same or different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Arrays can be fabricated by depositing (e.g., by contact- or jet-based methods) either precursor units (such as nucleotide or amino acid monomers) or pre-synthesized capture agent. An array is “addressable” when it has multiple regions of different moieties (e.g., different capture agents) such that a region (i.e., a “feature” or “spot” of the array) at a particular predetermined location (i.e., an “address”) on the array will detect a particular target. An “array layout” refers to one or more characteristics of the features, such as feature positioning on the support, one or more feature dimensions, and an indication of a moiety at a given location. Capture agents on the array are typically selected based on the sequences of the intended target analytes. Particular capture agents or strategies for designing capture agents are described in copending U.S. patent application Ser. No. 11/173,693 filed on Jul. 1, 2005 by Wang entitled “Nucleic Acid Probes for Analysis of Small RNAs and Other Polynucleotides” and the copending U.S. patent application filed on Oct. 21, 2005 by Wang et al. entitled “Analysis of microRNA” and designated attorney docket no. 10051514-1. “Interrogating” the array refers to obtaining information from the array, especially information about analytes binding to the array. “Hybridization assay” references a process of contacting an array with a mobile phase containing analyte. An “array support” refers to an article that supports an addressable collection of capture agents, and may be, e.g. an insoluble support, a planar support, or any other kind of support known in the microarray art. In particular embodiments, the support may be made of glass (e.g. a slide), plastic, metal, or other available material compatible with manufacture and use of the array.

The term “pre-determined” refers to an element whose identity is known prior to its use. For example, a “pre-determined analyte” is an analyte whose identity is known prior to any binding to a capture agent. An element may be known by name, sequence, molecular weight, its function, or any other attribute or identifier. In some embodiments, the term “analyte of interest”, i.e., a known analyte that is of interest, is used synonymously with the term “pre-determined analyte”.

Small RNA references RNAs less than about 300 bases long, generally less than about 200 bases long, e.g. less than about 100 bases long, less than about 60 bases long, less than about 50 bases long, less than about 40 bases long, or less than about 35 bases long. In particular embodiments, the small RNA may be selected from short interfering RNAs (siRNAs), microRNAs (miRNA), tiny non-coding RNAs (tncRNA) and small modulatory RNA (smRNA), or combinations thereof. See Novina et al., Nature 430: 161-164 (2004). In particular embodiments, small RNAs may be at least about 4 bases long, at least about 6 bases long, at least about 8 bases long, or longer.

Long polynucleotide references a polynucleotide that is separable from small RNAs using a size fractionation method as described herein or as known in the art to provide a fraction containing small RNA and another fraction containing other polynucleotides having lengths generally longer than about 300 bases. Long polynucleotides are polynucleotides generally longer than about 300 bases, e.g. longer than about 400 bases, longer than about 500 bases, longer than about 700 bases, and may be up to about 5000 bases long, up to about 10,000 bases long, or even longer.

A small RNA binding site refers to a sequence that specifically binds to a small RNA under conditions sufficient for the specific binding to occur. A small RNA binding site is typically complementary to the small RNA to which it binds.

“Complementary” references a property of specific binding between polynucleotides based on the sequences of the polynucleotides. As used herein, polynucleotides are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C. “Complementary” includes embodiments in which two polynucleotides are strictly complementary and also includes embodiments in which two polynucleotides are substantially complementary. In this regard, “strictly complementary” is a term used to characterize a first polynucleotide and a second polynucleotide, such as a target and a capture agent directed to the target, and means that every base in a sequence (or sub-sequence) of contiguous bases in the first polynucleotide has a corresponding complementary base in a corresponding sequence (or sub-sequence) of contiguous bases in the second polynucleotide. “Strictly complementary” means that there are no insertions, deletions, or substitutions in either of the first and second polynucleotides with respect to the other polynucleotide (over the complementary region). Put another way, every base of the complementary region may be paired with its complementary base, e.g. following normal base-pairing rules. “Substantially complementary” is a term used to characterize a first polynucleotide and a second polynucleotide, and means that there may be one or more relatively small (less than 10 bases, e.g. less than 5 bases, typically less than 3 bases, more typically a single base) insertions, deletions, or substitutions in the first and/or second polynucleotide (over the complementary region) relative to the other polynucleotide. The complementary region is the region that is complementary between a first polynucleotide and a second polynucleotide (e.g. a target analyte and a capture agent; further e.g. a small RNA and a small RNA binding site in a long polynucleotide such as a messenger RNA). Complementary sequences are typically embedded within larger polynucleotides, thus two relatively long polynucleotides may be complementary over only a portion of their total length. The complementary region is typically at least about 10 bases long, more typically at least about 12 bases long, more typically at least about 15 bases long, still more typically at least about 20 bases long, or may be at least about 25 bases long. In various typical embodiments, the complementary region may be up to about 200 bases long, or up to about 120 bases long, up to about 100 bases long, up to about 80 bases long, up to about 60 bases long, up to about 45 bases long, or up to about 40 bases long.

If a polynucleotide, e.g. a capture agent, is “directed to” a target, the polynucleotide has a sequence that is complementary to a sequence in that target and will specifically bind (e.g. hybridize) to that target under hybridization conditions. The hybridization conditions typically are selected to produce binding pairs of nucleic acids, e.g., capture agents and targets, of sufficient complementarity to provide for the desired level of specificity in the assay while being incompatible to the formation of binding pairs between binding members of insufficient complementarity to provide for the desired specificity. Such hybridization conditions are typically known in the art. Examples of such appropriate hybridization conditions are also disclosed herein for hybridization of a sample to an array. The target will typically be a small RNA, e.g. an miRNA, for embodiments discussed herein.

Accordingly, the invention thus relates to novel methods of performing an array analysis of an RNA sample. In certain embodiments, the invention provides a method of analyzing a sample containing small RNAs and other polynucleotides, wherein the other polynucleotides having small RNA binding sites. The sample is contacted with a size-fractionation medium under denaturing conditions to provide a fraction comprising small RNA. The fraction comprising small RNA is then contacted with an array under conditions sufficient to provide for specific binding to the array. The array includes an array substrate having capture agents bound thereto that are directed to small RNAs of interest. The array typically is then interrogated to provide data on binding of the small RNAs in the sample to the array.

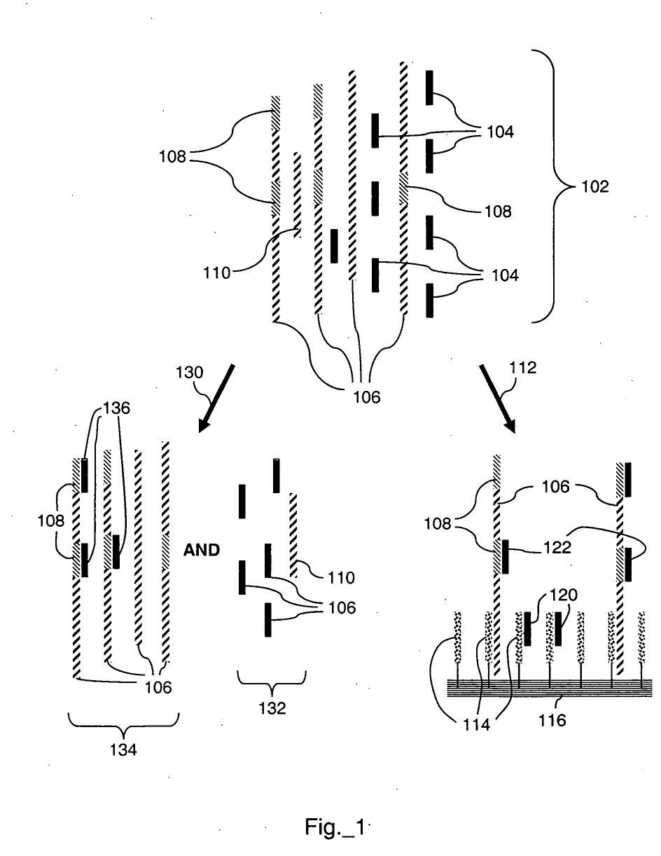

Without wishing to be bound to any particular theory of operation applied to any of the embodiments of the present invention, it is noted that the biological function of miRNA is presently believed to entail the binding of miRNA to regions of messenger RNA with partial or complete complementarity. It is reasonable to expect an abundance of miRNA binding sites within such messenger RNAs. These regions can result in undesired interference to quantitative measurements of miRNA as depicted in FIG. 1. The messenger RNAs are generally much longer than the microRNAs.

FIG. 1 illustrates a sample 102 that includes various polynucleotides. Illustrated in the sample 102 are small RNAs 104 (e.g. microRNAs) and other polynucleotides, such as long polynucleotides 106 (e.g. messenger RNAs). A portion of the long polynucleotides will include small RNA binding sites 108, i.e. sites that specifically bind to small RNAs 104 in the sample. As illustrated in FIG. 1, the sample 102 may further include other polynucleotides that are relatively short 110 compared to the long polynucleotides 106.

Still referring to FIG. 1, when the sample 102 is subjected to analysis 112 using a polynucleotide array, polynucleotides in the sample 102 will be bound to capture agents 114 immobilized on a surface of an array substrate 116, wherein each of the capture agents 114 is directed to a respective small RNA. Various instances of binding may occur on the array, including (i) binding of small RNAs (illustrated at 120) to complementary capture agents 114 (capture agents that are directed to the small RNAs); (ii) binding of other polynucleotides 106 via capture agents 114; and (iii) binding of small RNAs (illustrated at 122) to small RNA binding sites 108 that are part of the long polynucleotides 106. Typically, noise or erroneous results will be obtained while interrogating the array due to the binding described for instances (ii) and (iii), above.

To reduce such errors or noise in the results, the sample may be fractionated according to size. This is illustrated in FIG. 1, showing that the sample 102 is fractionated 130 based on size, to provide a first fraction 132 including small RNAs 104 and other polynucleotides that are relatively short 110 and a second fraction 134 containing long polynucleotides 106. FIG. 1 illustrates results of fractionating the sample under conditions that permit some small RNAs 136 from the sample to associate with the long polynucleotides 106 (e.g. via binding to small RNA binding sites 108 of the long polynucleotides 106) and hence to fractionate into the second fraction 134 containing the long polynucleotides 106. Thus, some of the small RNAs will be lost in the fractionating process, and subsequent array analysis of the first fraction 132 for small RNAs may potentially lead to lower sensitivity or failure to detect the small RNAs that partitioned into the second fraction 134.

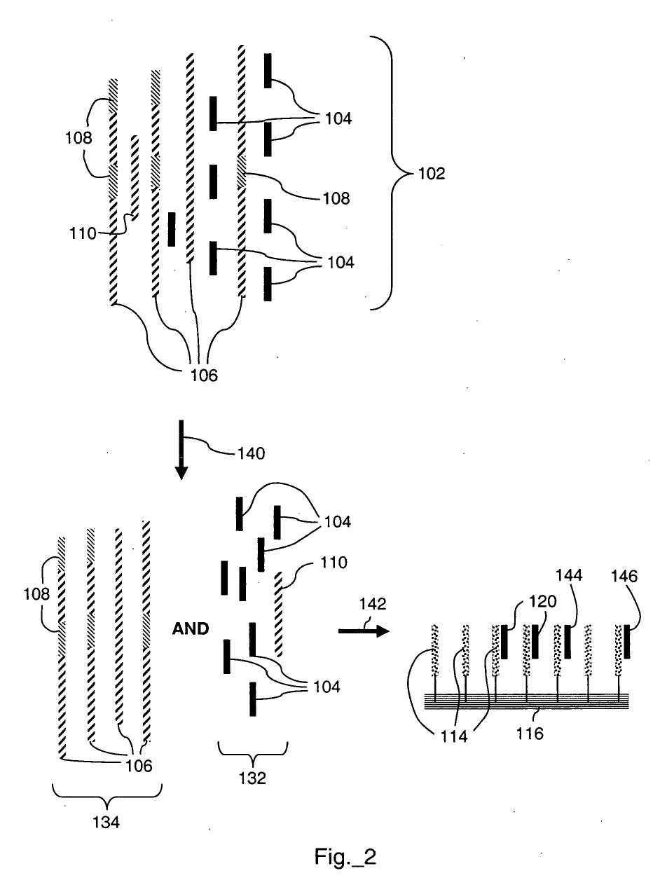

Therefore, FIG. 2 illustrates an embodiment in accordance with the present invention. The sample 102 includes various polynucleotides. Illustrated in the sample 102 are small RNAs 104 (e.g. microRNAs) and other polynucleotides, such as long polynucleotides 106 (e.g. messenger RNAs). A portion of the long polynucleotides will include small RNA binding sites 108, i.e. sites that may bind to small RNAs 104 in the sample under stringent assay conditions. As illustrated in FIG. 2, the sample 102 may further include other polynucleotides that are relatively short 110 compared to the long polynucleotides 106.

In an embodiment in accordance with the present invention, the sample is fractionated according to size under denaturing conditions. This is illustrated in FIG. 2, showing that the sample 102 is fractionated 140 to provide a first fraction 132 including small RNAs 104 and other polynucleotides that are relatively short 110 and a second fraction 134 containing long polynucleotides 106. The fractionation 140 occurs under denaturing conditions and fractionates the polynucleotides in the sample according to their size. Since the fractionation 140 occurs under denaturing conditions, the large polynucleotides 106 having small RNA binding sites 108 are preferentially separated from the small RNAs that are complementary to the small RNA binding sites 108. As a consequence, the polynucleotides in the sample 102 are more effectively separated according to their size, with less of the small RNAs being lost into the second fraction 134 (and instead being recovered in the first fraction 132).

Still referring to FIG. 2, when the first fraction 132 including the small RNA is subjected to analysis 142 using a polynucleotide array, small RNAs from the first fraction 132 will be bound to capture agents 114 immobilized on a surface of an array substrate 116, wherein each of the capture agents 114 is directed to a respective small RNA. As provided for in the present invention, the small RNAs bound to complementary capture agents 114 include small RNAs (illustrated at 120) that fractionate similarly under denaturing conditions versus non-denaturing conditions. In addition, the small RNAs bound to complementary capture agents 114 include additional small RNAs (illustrated at 144, 146) resulting from the denaturing size fractionation of the sample 102.

Thus, following size fractionation of the sample under denaturing conditions to obtain a first fraction containing small RNA, array analysis of the first fraction 132 may potentially lead to reduced losses of small RNA, where such losses are due to association with large polynucleotides.

It should be understood that “first fraction” and “second fraction” are terms merely indicating that a fractionation is conducted, and do not imply any particular sequence or limitation on the number of aliquots (i.e. “sub-fractions”) collected and pooled in a given fraction. Also, since the small RNA fraction (“first fraction”) is of primary interest, the second fraction may be a “remainder”, “flow-through”, or “adsorbed” fraction that is not collected or is not retained.

The subject invention provides a method of analyzing a sample for small RNA, e.g. assessing for the presence or amount of a miRNA. The sample typically includes polynucleotides having small RNA binding sites. In general, the method includes the following steps: a) contacting the sample with a size fractionation medium under denaturing conditions to provide a fraction comprising small RNA; b) contacting the fraction comprising small RNA with an array having capture agents directed to the small RNA; and c) interrogating the array to obtain information about small RNA in the sample. Contacting the array with the fraction comprising small RNA is typically performed under specific binding conditions, e.g. stringent assay conditions. Interrogating the array typically involves detecting the presence of any detectable label associated with the capture agents of the array, thereby evaluating the amount of the respective targets, e.g. small RNAs such as miRNAs, in the sample.

The sample of RNA may be obtained from any source. For example, the sample of RNA may be any RNA sample, typically a sample containing RNA that has been isolated from a biological source, e.g. any plant, animal, yeast, bacterial, or viral source, or a non-biological source, e.g. chemically synthesized. The sample may already be in solution form or may be a dried sample of RNA to which a reconstitution buffer is added. In particular embodiments, the sample of RNA includes one or more small RNAs, such as e.g. short interfering RNAs (siRNAs), microRNAs (miRNA), tiny non-coding RNAs (tncRNA) and small modulatory RNA (smRNA). See Novina et al., Nature (2004) 430: 161-164. In particular embodiments, the sample includes isolated small RNAs, e.g. the sample results from an isolation protocol for small RNA such as one or more of those listed in this paragraph. In certain embodiments, the small RNA targets may include isolated miRNAs, such as those described in the literature and in the public database accessible via the at the world-wide website of the Sanger Institute (Cambridge, UK) (which may be accessed by typing “www” followed by “.sanger.ac.uk/cgi-bin/Rfam/mirna/browse.pl” into the address bar of a typical internet browser). Methods for preparing samples of miRNAs from cells are well known in the art (see, e.g., Lagos-Quintana et al, Science 294:853-858(2001); Grad et al, Mol Cell 11:1253-1263 (2003); Mourelatos et al, Genes Dev 16:720-728(2002); Lagos-Quintana et al, Curr Biol 12:735-739(2002); Lagos-Quintana et al, RNA 9:175-179(2003) and other references cited above). In some embodiments, the sample of RNA may be a whole RNA fraction isolated from a biological source and includes messenger RNA and small RNA. Such samples including a diverse set of RNAs, such as a whole RNA fraction, may be referenced herein as “complex” RNA samples.

The method of the present invention includes separating components in the sample based on the molecular size of the components. In certain embodiments, the present invention provides an isolation protocol for small RNA, especially RNAs less than about 300 bases long, e.g. less than about 200 bases long, less than about 100 bases long, or less than about 50 bases long. The size-fractionation of the sample thus provides a fraction that includes isolated small RNAs. In particular embodiments, the size-based separation of the sample is performed by contacting the sample with a size fractionation medium under denaturing conditions and recovering a fraction containing the small RNAs that are of interest.

The separation can be performed using any media known in the art for performing molecular separations based on molecular weight (or molecular size) of the components of the sample being separated. Such media are referenced as “size fractionation media” herein. Various examples of a size fractionation medium include: a size-selectively permeable membrane (e.g. for size fractionation through the membrane, in which smaller species pass through the membrane and larger species cannot), a gel (e.g. a polyacrylamide gel for electrophoresis), a size exclusion chromatography medium, and others. In various embodiments, the size fractionation medium may be commercially available filters, such as those sold under the trademarks MICROCON™ or CENTRICON™ (by Millipore, Billerica, Mass.), or commercially available chromatography columns, such as those sold under the trademarks BIO-SPIN™ or MICROBIO-SPIN™ (by Bio-Rad Laboratories, Hercules, Calif.). Typical use of such size fractionation media follows instructions provided with the media or in published protocols which are well known.

In embodiments in which the size fractionation medium is a size-selectively permeable filter membrane, such as a MICROCON™ filter, the low molecular weight species will pass through the membrane and be collected in the collection tube, while the high molecular weight species (e.g. the long polynucleotides) will be retained in the membrane chamber. In embodiments in which the size fractionation medium is a size exclusion chromatography medium, such as a BIO-SPIN™ column, the small molecular weight species are retained in the medium, while the high molecular weight species (e.g. the long polynucleotides) are excluded (are not retained by the medium). The small molecular weight species are then washed, or eluted, from the medium and are collected. The small RNA is recovered from the media, e.g. by elution via further washing of the media or via change in buffer composition. The recovered eluate generally undergoes buffer exchange and concentration before proceeding with the hybridization reaction on the array.

In certain embodiments, contacting the sample with the size fractionation medium includes selecting polynucleotides less than about 300 bases long (e.g. less than about 200 bases long, less than about 100 bases long, less than about 50 bases long) to provide the fraction comprising the small RNA. As an example, this may be done by collecting the eluate from the size fractionation medium in aliquots, and then pooling selected aliquots to provide the fraction comprising the small RNA.

In particular embodiments, in the fraction comprising the small RNAs, polynucleotides shorter than about 300 bases constitute at least 5% (e.g. at least 10%, at least 20%, at least 40%, at least 60%, at least 80%, at least 90%, at least 95%) of the total polynucleotides in the fraction. This percentage is calculated as: (mass of polynucleotides less than about 300 bases long in a given volume of the fraction comprising the small RNAs) divided by (total mass of polynucleotides in the given volume of the fraction comprising the small RNAs), and then expressed as a percentage. In various embodiments, at least about 10% (e.g. at least about 20%, 40% or 60%) of the polynucleotides in the fraction comprising the small RNAs are shorter than about 300 bases, e.g. generally shorter than about 200 bases, shorter than about 100 bases long.

In certain embodiments, in the fraction comprising the small RNAs, long polynucleotides constitute less than about 80% (e.g. less than about 60%, less than about 40%, less than about 20%, less than about 10%) of the total polynucleotides in the fraction comprising the small RNAs. This percentage is calculated as: (mass of long polynucleotides in a given volume of the fraction comprising the small RNAs) divided by (total mass of polynucleotides in the given volume of the fraction comprising the small RNAs), and then expressed as a percentage. In various embodiments, less than about 60% (e.g. less than about 40%, 20% or 10%) of the polynucleotides in the fraction comprising the small RNAs are longer than about 300 bases long, or are longer than about 400 bases long, or are longer than about 500 bases long. In certain embodiments, polynucleotides longer than about 300 bases (e.g. longer than about 400 bases, longer than about 500 bases) make up an insubstantial amount of the fraction comprising the small RNAs. In this regard, “an insubstantial amount” is an amount which does not substantially interfere with binding of small RNAs to the capture agents of the array, i.e. less than about 5% error is introduced into the binding measurements obtained during interrogation of the array due to the presence of the long polynucleotides.

Contacting the sample with the size fractionation medium is done under denaturing conditions. As used herein, for a solution containing complementary polynucleotides, denaturing conditions are conditions sufficient to result in dissociation of intermolecular duplexes formed between the complementary polynucleotides. Denaturing conditions are conditions which also typically reduce or prevent the formation of intermolecular duplexes between complementary polynucleotides. Therefore, under denaturing conditions, substantially all intermolecular duplexes among polynucleotides in the sample are dissociated. In this context, “substantially all” means that, of the nucleotides in the sample that are capable forming intermolecular duplexes with other nucleotides in the sample, at least 60% (e.g. at least 70%, at least 80%, at least 90%, at least 95%) are not in an intermolecular duplex conformation. In particular embodiments, under denaturing conditions, substantially all polynucleotides (including small RNAs) in the sample are in single-stranded conformation. Denaturing conditions typically reduce the tendency of small RNAs to associate with long polynucleotides during the separation.

The denaturing conditions should be compatible with the size fractionation medium used, e.g. a denaturant that is added to the sample should be chemically compatible with the chosen separation method. Denaturants used for disrupting binding between complementary strands of polynucleotides are well known and include, e.g. urea, guanidine salts, formamide, DMSO, formaldehyde, glyoxal, methyl mercuric hydroxide, 2-pyrrolidone, or combinations thereof. Denaturing conditions may also include conditions of temperature, ionic strength, buffer composition, etc., that result in destabilization of double stranded duplex structures of polynucleotides. For example, the sample can be heated to enhance denaturation and then flash cooled. Denaturing conditions are generally known in the art.

In particular embodiments, a denaturant is added to the sample, typically at a concentration that discourages aggregation between the target small RNAs and other components of the sample. To achieve sufficient denaturation of the polynucleotides (including small RNAs) in the sample the concentration of denaturant may be adjusted based on routine experimentation given the disclosure herein. Other experimental parameters may be selected based on known ranges for the experimental parameters or determined through routine experimentation. Such other experimental parameters may include, e.g. temperature, pH, ionic strength, source of RNA, structural feature of RNA, concentration of RNA, presence of other materials (e.g. contaminants, salt, surfactant, other solvents) etc.

In particular embodiments, a typical protocol for the size-based fractionation of the sample under denaturing conditions proceeds as follows: The RNA sample is dissolved in buffer (pH 6.7-8.5) to give a concentration in the range of about 1 pg/ml solution to about 1 μg/ml solution. If Tris buffer is used, the pH at room temperature is preferably above 7. A typical buffer has low or no magnesium and contains EDTA (usually 1-5 mM). The buffer also contains a denaturant, such as urea at a concentration of about 1M to about 8M. Additional denaturant such as formamide (1-99%), DMSO (1-99%), formaldehyde (3% w/v), glyoxal (10-30%), methyl mercuric hydroxide (3-5 mM), 2-pyrrolidone (5%) could be used in addition to or instead of urea. Further, the buffer can contain surfactants such as SDS (0.01-10%) or TRITON™ surfactant. The sample is thoroughly mixed, e.g. using a vortex mixer. Preferably, samples should be heated to 45-105° C. for 1-2 min/10 μl solution and immediately cooled in ice water or room temperature water. Ice water is typically used, although room temperature water may be used if needed to prevent precipitation or freezing of the sample/buffer mixture. Alternatively samples can be kept at 20-45° C. (if evaporation is not a problem) throughout the separation process after heating. The denatured solution is then passed through a filtering device such as MICROCON™ YM-100 filter (or media compatible with the denaturing conditions). Collect the low molecular weight fraction for miRNA (and/or other small RNA). If MICROCON™ YM-100 filter is used, the flow-through should be collected. The recovered fraction may then be contacted with an array to perform a hybridization assay, as described herein.

In particular embodiments, the recovered fraction of small RNAs is labeled prior to being contacted with the array. In certain embodiments, the sample may be labeled prior to the size fractionation step, or the sample may be isolated from a source already labeled. Whether it is the sample that is labeled or the recovered fraction that is labeled, the result is a population of labeled small RNAs, e.g. miRNAs. In this regard, where the discussion below refers to labeling a sample, it should be understood that the labeling may, in certain embodiments and where context permits, be performed on the fraction of small RNA recovered from the size fractionation (rather than the initial sample). In typical embodiments, binding of labeled small RNA to the capture agents of the array is detected by detecting the label associated with the capture agents (due to binding of the labeled small RNAs). In general, labeling methods are well known in the art (e.g., using RNA ligase, polyA polymerase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.), and, accordingly, such methods do not need to be described here in great detail. The observable label may be any observable label known in the art, e.g. a chromophore, a fluorescent label, a spin label, a radioisotope label, a mass label, a sequence label, a chemically reactive tag, an affinity label, or any other known label. In particular embodiments, the label is a fluorescent dye, which labels will be described in greater detail below.

Fluorescent dyes of particular interest include: xanthene dyes, e.g. fluorescein and rhodamine dyes, such as fluorescein isothiocyanate (FITC), 6 carboxyfluorescein (commonly known by the abbreviations FAM and F), 6 carboxy-2′,4′,7′,4,7-hexachlorofluorescein (HEX), 6 carboxy 4′,5′ dichloro 2′,7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarins, e.g. umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc. More information about commercially available dyes for oligonucleotide conjugation can be found at the Synthegen website (which may be accessed by typing “www” followed by “.synthegen.com” into the address bar of a typical internet browser). Any such dyes may potentially be used in accordance with the methods described herein. Such labels typically are well known in the art.

In certain embodiments, binding of labeled small RNAs is assessed with respect to binding of at least one labeled control sample. In one example, a suitable labeled control sample may be made from a control cell population, as will be described in greater detail below. In certain embodiments, a sample and a control sample may be prepared and labeled, and relative binding of the labeled small RNAs from the samples to capture agents on an array may be assessed. Typically, the labeled small RNAs are contacted with the array under stringent hybridization conditions.

In practicing the subject methods, the sample and control sample may be labeled to provide at least two different populations of labeled small RNAs that are to be compared. The populations of small RNAs may be labeled with the same label or different labels, depending on the actual assay protocol employed. For example, where each population is to be contacted with different but identical arrays, each population of small RNAs may be labeled with the same label. Alternatively, where both populations are to be simultaneously contacted with a single array of surface-bound capture agents, i.e., co-hybridized to the same array of immobilized capture agents, the two different populations are generally distinguishably labeled with respect to each other.

The samples are sometimes labeled using “distinguishable” labels in that the labels that can be independently detected and measured, even when the labels are mixed. In other words, the amounts of label present (e.g., the amount of fluorescence) for each of the labels are separately determinable, even when the labels are co-located (e.g., in the same tube or in the same duplex molecule or in the same feature of an array). Suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexafluor555 and Alexafluor647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), fluorescein and Texas red (Dupont, Boston Mass.) and POPRO3 and TOPRO3 (Molecular Probes, Eugene, Oreg.). Further suitable distinguishable detectable labels may be described in Kricka et al. (Ann. Clin. Biochem. 39:114-29, 2002).

In certain embodiments, at least a first population of small RNAs and a second population of small RNAs are produced from two different small RNA-containing samples, e.g., two populations of cells. As indicated above, depending on the particular assay protocol (e.g., whether both populations are to be hybridized simultaneously to a single array or whether each population is to be hybridized to two different but substantially identical, if not identical, arrays) the populations may be labeled with the same or different labels. As such, a feature of certain embodiments is that the different populations of small RNAs are labeled with the same label such that they are not distinguishably labeled. In yet other embodiments, a feature of certain embodiments is that the different populations of small RNAs are labeled with different labels such that they are distinguishable from each other.

Accordingly, in typical embodiments the subject methods include a hybridization assay that typically includes the following steps: (1) providing an array having capture agents bound to an array support; (2) contacting a population of labeled small RNAs with the capture agents on the array support under conditions sufficient to provide for specific binding, e.g. typically under stringent hybridization conditions; (3) post-hybridization washes to remove nucleic acids not bound in the hybridization; and (4) detection of the hybridized small RNAs. The reagents used in each of these steps and their conditions for use may vary depending on the particular application.

As indicated above, hybridization assay is carried out under suitable hybridization conditions, which may vary in stringency as desired; typical conditions are sufficient to produce capture agent/target complexes on an array surface between complementary binding members, e.g., between surface-bound capture agents and labeled complementary small RNAs. In certain embodiments, stringent hybridization conditions may be employed. Representative stringent hybridization conditions that may be employed in these embodiments are provided above.

In typical embodiments, after a labeling reaction to label polynucleotides (e.g. target small RNAs) and after size fractionation of polynucleotides (possibly, though not necessarily, in that order), the fraction containing the small RNAs is contacted with an array having capture agents bound to a support, as discussed above, under conditions such that nucleic acid hybridization to the capture agents can occur, e.g., in a buffer containing 50% formamide, 5×SSC and 1% SDS at 42° C., or in a buffer containing 5×SSC and 1% SDS at 65° C., both with a wash of 0.2×SSC and 0.1% SDS at 65° C., for example.

The above hybridization step may include agitation of the array and the fraction containing the labeled small RNA, where the agitation may be accomplished using any convenient protocol, e.g., shaking, rotating, spinning, and the like.

Standard hybridization techniques (e.g. under conditions sufficient to provide for specific binding of small RNA, e.g. target miRNAs, to the capture agents on the array) are used to hybridize a solution containing polynucleotides (e.g. small RNAs) to a nucleic acid array. Suitable methods are described in many references (e.g., Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). See also U.S. Pat. Nos. 6,335,167; 6,197,501; 5,830,645; and 5,665,549; the disclosures of which are herein incorporated by reference. The hybridization is typically performed under stringent hybridization conditions, as described herein and as known in the art. Selection of appropriate conditions, including temperature, salt concentration, polynucleotide concentration, time(duration) of hybridization, stringency of washing conditions, and the like will depend on experimental design, including source of sample, identity of capture agents, degree of complementarity expected, etc., and may be determined as a matter of routine experimentation for those of ordinary skill in the art.

Following hybridization, the array is typically washed to remove unbound nucleic acids. Washing may be performed using any convenient washing protocol, where the washing conditions are typically stringent, as described above.

Following hybridization and washing, as described above, the hybridization of target analytes (e.g. small RNAs) to the capture agents is then detected using standard techniques of reading the array, i.e. the array is interrogated. Reading the resultant hybridized array may be accomplished by illuminating the array and reading the location and intensity of resulting fluorescence at each feature of the array to detect any binding complexes (e.g. capture agent/target analyte duplexes) on the surface of the array. For example, a scanner may be used for this purpose that is similar to the AGILENT MICROARRAY SCANNER available from Agilent Technologies, Palo Alto, Calif. Other suitable devices and methods are described in U.S. Pat. No. 6,756,202 and U.S. Pat. No. 6,406,849. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from interrogating the array may be raw results (such as fluorescence intensity readings for each feature in one or more color channels) or may be processed results (such as those obtained by subtracting a background measurement, or by rejecting a reading for a feature which is below a predetermined threshold, normalizing the results, calculating log ratios for the results, and/or forming conclusions based on the pattern read from the array (such as whether or not a particular target miRNA may have been present in the sample, or whether or not a pattern indicates a particular condition of an organism from which the sample came).

By “normalization” is meant that data corresponding to the two populations of polynucleotides are globally normalized to each other, and/or normalized to data obtained from controls (e.g., internal controls produce data that are predicted to be equal in value in all of the data groups). Normalization generally involves multiplying each numerical value for one data group by a value that allows the direct comparison of those amounts to amounts in a second data group. Several normalization strategies have been described (Quackenbush et al, Nat. Genet. 32 Suppl: 496-501, 2002, Bilban et al Curr Issues Mol Biol. 4:57-64, 2002, Finkelstein et al, Plant Mol Biol. 48(1-2):119-31, 2002, and Hegde et al, Biotechniques. 29:548-554, 2000). Specific examples of normalization suitable for use in the subject methods include linear normalization methods, non-linear normalization methods, e.g., using lowest local regression to paired data as a function of signal intensity, signal-dependent non-linear normalization, qspline normalization and spatial normalization, as described in Workman et al., (Genome Biol. 2002 3, 1-16). In certain embodiments, the numerical value associated with a feature signal is converted into a log number, either before or after normalization occurs. Data may be normalized to data obtained using a support-bound polynucleotide capture agent directed to a particular control polynucleotide, where the control polynucleotide is included in the hybridization at a known concentration, for example.

In certain embodiments, results from interrogating the array are used to assess the level of binding of the small RNAs from the sample to capture agents on the array. The term “level of binding” means any assessment of binding (e.g. a quantitative or qualitative, relative or absolute assessment), usually done, as is known in the art, by detecting signal (i.e., pixel brightness) from a label associated with the small RNA, e.g. the sample is labeled. The level of binding of labeled small RNA to capture agent is typically obtained by measuring the surface density of the bound label (or of a signal resulting from the label).

Accordingly, since the arrays used in the subject assays may contain capture agents for a plurality of different small RNAs, the presence of a plurality of different small RNAs in a sample may be assessed. The subject methods are therefore suitable for simultaneous assessment of a plurality of small RNAs in a sample.

In certain embodiments, a surface-bound capture agent may be assessed by evaluating its binding to two populations of small RNAs that are distinguishably labeled. In these embodiments, for a single surface-bound capture agent of interest, the results obtained from hybridization with a first population of labeled small RNAs may be compared to results obtained from hybridization with the second population of small RNAs, usually after normalization of the data. The results may be expressed using any convenient means, e.g., as a number or numerical ratio, etc.

Accordingly, in typical embodiments, after recovery of a fraction containing small RNA, as described above, the recovered fraction containing the small RNA is then labeled, e.g. with Cy5 or Cy3, and hybridized onto an array as follows: The recovered fraction containing the small RNA is desalted (e.g. with BioRad MICRO BIO-SPIN™-6 columns, as directed by BioRad instructions) to remove excess observable label remaining from the labeling reaction. The desalted fraction containing the small RNA is added to solution containing water and carrier (25-mer DNA with random sequence). The resulting solution is heated at about 100° C. for approximately 1 minute per 10 microliters of solution, and then immediately cooled on ice. The cooled solution is then added to hybridization buffer and mixed carefully. The final solution is then contacted with the array, e.g. in a SUREHYB™ hybridization chamber (Agilent Part Number: G2534A), and placed on the rotisserie of a hybridization oven overnight. The hybridization temperature is typically in the range from about 50° C. to about 65° C., or in the range from about 55° C. to about 60° C., although temperatures outside this range (e.g. in the range from about 30° C. to about 65° C., or in the range from about 45° C. to about 65° C.) may be used depending on the other experimental parameters, e.g. hybridization buffer composition and wash conditions. After the hybridization is complete, the array is washed thoroughly and dried with nitrogen as needed. The array is scanned (e.g. with an Agilent Scanner, Agilent Product Number: G2565BA). The data is then evaluated (e.g. using Agilent Feature Extraction Software, Agilent Product Number: G2567AA) for hybridization efficiency and specificity. Data may be further analyzed, e.g. using Spotfire software and Microsoft Excel.

Also provided by the subject invention are kits for practicing the subject methods, as described above. The subject kits include at least an array having capture agents directed to target small RNAs, e.g. target miRNAs. For example, a kit may include such an array and a size fractionation medium, as described above. In certain embodiments the subject kits may also include reagents for isolating small RNA from a source (e.g. a tissue, cell, tissue lysate) to provide an isolated sample of small RNA. In some embodiments the subject kits optionally also include one or more constituents selected from: reagents employed in conjunction with the size fractionation medium for fractionating the sample; reagents for labeling RNA; reagents for contacting the sample of RNA with the array. Examples of such constituents may include (but are not limited to), e.g., enzymes for use with the subject methods such as described above, control samples, reagents for performing an array hybridization, combinations thereof, etc. The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a suitable material, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable material.

The practice of the present invention will employ, unless otherwise indicated, conventional techniques of synthetic organic chemistry, biochemistry, molecular biology, and the like, which are within the skill of the art. Such techniques are explained fully in the literature. Unless otherwise defined herein, all technical and scientific terms used herein have the same meaning as commonly understood by one of ordinary skill in the art to which the invention belongs. The description herein is put forth so as to provide those of ordinary skill in the art with a complete disclosure of the methods and compositions disclosed and claimed herein. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. Standard temperature and pressure are defined as 20° C. and 1 atmosphere.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties, provided that, if there is a conflict in definitions, the definitions provided herein shall control.

What is claimed is:

1. A method of analyzing a sample comprising small RNAs and other polynucleotides, the other polynucleotides having small RNA binding sites, the method comprising:
a) contacting the sample with a size fractionation medium under denaturing conditions to provide a fraction comprising the small RNAs; and
b) contacting the fraction comprising the small RNAs with an array having capture agents directed to the small RNAs, said contacting with the array performed under conditions sufficient to provide for binding of the small RNAs to the capture agents, and
c) interrogating the array to obtain information about the small RNAs in the sample.

2. The method of claim 1, wherein contacting the sample with the size fractionation medium includes selecting polynucleotides less than about 300 bases long to provide the fraction comprising the small RNA.

3. The method of claim 1, wherein contacting the sample with the size fractionation medium includes selecting polynucleotides less than about 100 bases long to provide the fraction comprising the small RNA.

4. The method of claim 1, wherein the size fractionation medium is selected from a size-selectively permeable membrane, a gel, a size exclusion chromatography medium.

5. The method of claim 1, wherein polynucleotides shorter than about 300 bases constitute at least 5% of the total polynucleotides in the fraction comprising the small RNA.

6. The method of claim 1, wherein polynucleotides shorter than about 300 bases constitute at least 40% of the total polynucleotides in the fraction comprising the small RNA.

7. The method of claim 1, wherein said small RNAs are selected from the group consisting of a short interfering RNA (siRNA), microRNA (miRNA), tiny non-coding RNA (tncRNA), a small modulatory RNA (smRNA), and combinations thereof.

8. The method of claim 1, wherein the small RNAs comprise microRNAs

9. The method of claim 1, wherein said denaturing conditions include a denaturant selected from the group consisting of urea, guanidine salts, formamide, DMSO, formaldehyde, glyoxal, methyl mercuric hydroxide, 2-pyrrolidone and combinations thereof, said denaturing conditions effective to dissociate substantially all intermolecular duplexes among polynucleotides in the sample.

10. The method of claim 1, wherein the array comprises at least 20 different capture agents, each of the capture agents directed to a different small RNA.

11. The method of claim 1, wherein the array comprises at least 20 different capture agents, each of the capture agents directed to a different microRNA.

12. The method of claim 1, wherein said conditions sufficient to provide for binding of the small RNAs to the capture agents include stringent hybridization conditions.

13. The method of claim 1, further comprising, prior to contacting the fraction comprising the small RNAs with the array, labeling the small RNAs with an observable label.

14. The method of claim 1, further comprising labeling the small RNAs and other polynucleotides in the sample with an observable label.

15. The method of claim 1, wherein the small RNAs in the fraction comprising the small RNAs have an observable label.

16. A kit for analyzing a sample for small RNAs, the sample comprising polynucleotides having small RNA binding sites, the kit comprising:
an array comprising capture agents bound to an array support, the capture agents directed to said small RNAs; and
a size fractionation medium.

17. The kit of claim 16, further comprising instructions for performing a method according to claim 1 using the array and the size fractionation medium.

18. The kit of claim 16, further comprising reagents for isolating polynucleotides from a cell.

19. The kit of claim 16, further comprising labeling reagents for labeling said polynucleotide.

20. The kit of claim 16, further comprising a hybridization buffer for use with the array.
